(19) United States
(12) Patent Application Publication
(10) Pub. No.: US 2021/0212862 A1
(43) Pub. Date: Jul. 15, 2021

(54) Compression Bandaging

(71) Applicant: Andover Healthcare, Inc., Salisbury, MA (US)

(72) Inventors: Thomas S. Murphy, Salisbury, MA (US); James Boyle, Salisbury, MA (US)

(21) Appl. No.: 17/265,540

(22) PCT Filed: Aug. 21, 2019
(86) PCT No.: PCT/US2019/047506

§ 371 Date: Feb. 3, 2021

Related U.S. Application Data
(60) Provisional application No. 62/721,222, filed on Aug. 22, 2018.

(51) Int. Cl. A61F 13/00; A61K 33/30; A61K 33/26; A61K 47/32; A61L 15/44; A61K 36/886; A61K 31/05; A61K 31/351; A61K 36/185; A61K 36/61; A61K 31/7056; A61K 31/122; A61K 31/4174; A61K 31/496; A61K 31/137; A61K 31/225; A61K 35/04; A61K 31/69; A61K 45/06; A61K 31/522; A61K 31/513; A61K 31/196; A61K 31/728; A61K 31/437; A61K 31/222; A61K 39/395; A61L 15/24; A61F 13/08

(52) U.S. Cl. CPC A61F 13/00063; A61L 2300/204; A61K 33/26; A61K 47/32; A61L 15/44; A61K 36/886; A61K 31/05; A61K 31/351; A61K 36/185; A61K 36/61; A61K 31/7056; A61K 31/122; A61K 31/4174; A61K 31/496; A61K 31/137; A61K 31/225; A61K 35/04; A61K 31/69; A61K 45/06; A61K 31/522; A61K 31/513; A61K 31/196; A61K 31/728; A61K 31/437; A61K 31/222; A61K 39/39558; A61L 15/24; A61F 13/08; A61F 2013/00272; A61F 2013/00285; A61L 2300/102; A61L 2300/30; A61L 2300/216; A61L 2300/232; A61L 2300/236; A61K 33/30

(57) ABSTRACT

A compression bandage is provided that includes a zinc oxide composition and/or one or more other therapeutic agents. In use, the bandage can be compression wrapped directly contacting a subjects skin for application of the zinc oxide composition and/or other therapeutic agents.

### FIELD

The present invention relates generally to a compression bandage that includes a zinc oxide composition and/or one or more other therapeutic agents. In use, the bandage can be compression wrapped directly contacting a subject's skin for application of the zinc oxide composition and/or other therapeutic agents.

### BACKGROUND

Compression bandages are frequently used in medical and sports applications requiring a strong and reliable, yet comfortable and easily applied, means of securing a limb or other body segment for prolonged periods of time. For example, strains and sprains can cause inflammation and the accompanying accumulation of fluid around a sprained joint. Wrapping the affected joint securely with an elastic bandage can prevent excess fluid from accumulating and causing additional tissue damage.

Certain tapes and bandages are available for such applications.

It would be desirable to have improved compression bandages.

### SUMMARY

We now provide new compression-type elastic bandages that include one or more agents for transdermal contact or administration to a subject. The present bandages can be suitably used as the sole bandage or material to treat a subject.

More particularly, in a first preferred aspect, a compression bandage is provided that comprises a stretchable, wrappable bandage material for contact on a subject's skin, where the bandage material comprises a zinc oxide composition. Calamine is a preferred zinc oxide composition.

In a preferred further aspect, a compression bandage is provided that comprises a stretchable, wrappable bandage material for contact on a subject's skin, where the bandage material comprises one or more therapeutic agents other than a calamine composition, such as one or more antibiotics, antibacterial agents, antimicrobial agents, anti-odor agents, anti-inflammatory agent, anti-fungal agents, anti-viral agents, anti-cancer agents (including for treatment of a skin cancer and skin pre-cancers), eczema or psoriasis treatment agents, pain relieving agents, or combinations thereof. Preferably, such agents can be effectively administered transdermally to a subject.

In certain embodiments, the zinc oxide composition and/or therapeutic agents are present substantially throughout the bandage material, for example impregnated within and/or on the bandage material.

In certain other embodiments, the zinc oxide composition and/or therapeutic agents may be present in selected regions of the bandage material, for example within one or more strip regions extending the length and/or width of the bandage material where the zinc oxide and/or therapeutic agents do not reside outside the strip regions.

In each of those configurations, preferably the integration with the bandage material is such that the zinc oxide composition and/or therapeutic agents can readily contact and be administered to a subject skin upon application (for compression wrapping) of the bandage.

The compression bandage may be formed from a variety of materials and suitably may be a stretchable polymer composition. Suitable bandage materials include those that contain latex as well as those that are substantially or completely latex-free.

The compression bandage is suitably stretchable to facilitate applying a compressive force on a subject during use, i.e. a subject's limb, torso, head can be wrapped with the bandage and a compressive force thereby applied. In certain embodiments, the bandage material may have a maximum stretched length that is at least 30, 40, 50, 60, 70, 80, 90 or 100% or more greater than the material's unstretched length. In some preferred materials, the bandage material may have a maximum stretched length that is at least 40 to 80, 90 or 100% or more greater than the material's unstretched length.

The present compression bandages may be wound into a roll, e.g., for convenient storage and transport prior to use.

Methods for treating a subject are also provided which suitably include circumferentially wrapping a subject with a compression bandage as disclosed herein, where the bandage directly contacts subject's skin. Thus, in these preferred methods, a cushioned or absorbent material, or other material is not utilized and is thus not interposed between the present compression bandage and the subject's skin. Rather, the compression bandage is applied directly on and directly contacts the subject's skin. In certain applications, it may be desirable to apply another distinct material (e.g. a sleeve or stocking layer) over the wrapped compression bandage.

A wide variety of subjects suitably may be identified to utilize the present compression bandage. Thus, for instance, a person suffering from a sprain, strain or bruise, e.g. a joint or muscle sprain, strain or bruise, may experience relief by the compression force provided by a wrapped bandage as disclosed herein. Additionally, a zinc oxide composition, particularly calamine, present with the bandage can provide a favorable sensation to the subject's skin. Other therapeutic agents that may be present with the bandage also can provide a favorable effect for the subject. In particular, such one or more other therapeutic agents may be effectively administered transdermally to the subject.

In certain aspects, the subject will not be suffering from, at least in the skin area on which the compression bandage is applied, from any type of wound, skin ulcer, cancer (such as skin cancer or skin pre-cancer) psoriasis, eczema, infection, or the like.

In other aspects, the subject may have one or more of such a wound, skin ulcer, psoriasis, eczema, cancer (including skin cancer or skin pre-cancer), infection, or the like, and the present compression bandage is applied directly on the injury or distressed skin area.

Kits are also provided that contain a compression bandage as disclosed herein. Suitably, a kit also may contain written materials for use and/or identification of the compression bandage. The compression bandage is suitably packaged in roll form, i.e. where a strip of the bandage such as between 1 and 6 or more inches wide, such as 1, 2, 3, 4, 5 or 6 inches wide is wound and stored as a roll such as in lengths of at least one yard, more typically 2, 3, 4, 5, 6, 7, 8, 9, 10 yards or more.

As referred to herein, the term zinc oxide containing composition is intended to include any composition containing zinc oxide as an active ingredient and which is suitable for impregnating the bandage material. The zinc oxide containing compositions herein may include calamine, ichthammol, or both. In certain aspects, the zinc oxide containing compositions may optionally include an antimicrobial agent and/or may be used in the presence of or the absence of an antimicrobial agent. If an antimicrobial agent is present, either as a part of the zinc oxide containing composition or as a separately applied antimicrobial agent or formula, such antimicrobial agent may be any antimicrobial agent suitable for topical administration, including organic or inorganic antimicrobial agents.

Terms such as stretchable bandage, elastic bandage, elastic layer, compression bandage, compression layer, and similar terms are intended to encompass both short stretch bandages and long stretch bandages unless specifically stated otherwise.

Other aspects of the invention are discussed infra.

### DETAILED DESCRIPTION

As discussed, new compression-type elastic bandages are provided that are adapted for direct skin contact. In preferred aspects, in use on a subject's leg (e.g. ankle, foot, lower leg, knee, upper leg), torso, arm (e.g. hand, forearm, elbow, upper arm), neck, or head, the bandage is circumferentially wrapped around the selected region of the subject, and the bandage directly (no interposing material) contacts the subject's skin for the complete circumference of that region of the subject, e.g. the complete circumference of the subject's leg (e.g. any of ankle, foot, lower leg, knee, upper leg), torso, arm (e.g. any of hand, forearm, elbow, upper arm), neck, or head,

The bandage material comprises one or more agents for contact to a subject's skin.

A zinc oxide composition, particularly calamine, is a preferred agent incorporated with a bandage material for contact to a subject's skin. Calamine may include zinc oxide and ferric oxide.

Additional preferred agents incorporated with a bandage material for contact to a subject's skin include one or more of antibiotic, an antibacterial agent, an antimicrobial agent, an anti-odor agent, an anti-inflammatory agent, an anti-fungal agent, an anti-viral agent, an anti-cancer agent (including for treatment of a skin cancer and skin pre-cancers), an eczema or psoriasis treatment agent, a pain relieving agent, or combination thereof.

More specifically, preferred additional agents incorporated with a bandage material for contact to a subject's skin include one or more of aloe; menthol; mupirocin; witch hazel, tree tea oil; clindamycin; anthralin; clotrimazole; ketoconazole; terbinafine; benzoyl peroxide; coal tar; corticosteroids; crisaborole; retinoids; salicylic acid; acyclovir; 5-fluorouracil; 5-fluorouracil, diclofenac; hyaluronic acid (alone or together with diclofenac); imiquimod; ingenol mebutate; adalimumab; and eucrisa.

Suitable anti-odor agents incorporated with a bandage material for contact to a subject's skin include, among others, activated carbon, cyclodextrins/modified cyclodextrins, activated alumina, metal powders, alumina silicates, metal oxides, zeolites, ceramics, diatomaceous earth, macroporous polymers, aerogels, cellulose and cellulosic derivatives, starches and starch derivatives, clay, talc, sodium bicarbonate, silicon dioxide, and combinations thereof.

Suitable antimicrobial agents incorporated with a bandage material for contact to a subject's skin include, among others, any appropriate antimicrobial composition useful for the intended purpose of preventing or inhibiting the growth or reproduction of microbes, such as bacteria, fungi, viruses , or protozoa, for example, selected from Beta Hemolytic Streptococci (Streptococcus pyogenes), Enterococci (Enterococcus faecalis), Staphylococci (Staphylococcus aureus/MRSA), Pseudomonas aeruginosa, Enterobacter species, Escherichia coli, Klebsiella species, Proteus species, Bacteroides species, Clostridium species, Candida species, A spergillus species. In preferred embodiments, the antimicrobial agent may be inorganic metal based/organic antimicrobial agents or the like, although it will be recognized that the antimicrobial agent may other antimicrobial agents as known in the art, including antibiotics, antiseptics, antiviral agents, antifungal agents, and disinfectants. In especially preferred embodiments, the antimicrobial agent is silver nanoparticles or silver nitrate.

Suitable antibiotic and antibacterial agents incorporated with a bandage material for contact to a subject's skin include, among others Methicillin, Neomycin sulfate, Bacitracin, Neomycin sulfate, and polymyxin B sulfate.

In certain preferred embodiments, the bandage material will comprise one or more agents for treatment of skin cancers and skin pre-cancers such as hypertrophic solar keratoses (including dysplastic hypertrophic solar keratosis and bowenoid hypertrophic solar keratosis), actinic keratosis and squamous cell carcinoma. In particular, for treatment of such skin cancers and pre-cancers, the bandage material may include one or more of 5-fluorouracil, diclofenac and/or hyaluronic acid, imiquimod and ingenol mebutate.

In certain methods of the invention, a subject suffering from or susceptible to a particular disease or disorder will be identified and selected to use a particular compression bandage.

For instance, a subject that has suffered from a strain or sprain (such as a strain or sprain of an ankle, elbow, knee, lower leg, upper leg, forearm, upper arm and the like) may be identified and selected, and that identified and selected subject's sprained/strained area may be wrapped with a compression bandage comprising calamine as disclosed herein.

A subject that is suffering from a skin cancer or pre-cancer such as hypertrophic solar keratoses (including dysplastic hypertrophic solar keratosis and bowenoid hypertrophic solar keratosis), actinic keratosis and/or squamous cell carcinoma may be identified and selected, and a compression bandage comprising one or more anticancer agents may be wrapped around the skin cancer regions of the selected subject. The one or more anticancer agents present in the compression bandage may be for example 5-fluorouracil, diclofenac and/or hyaluronic acid, imiquimod and ingenol mebutate.

Preferred bandage materials may be in strip or tape form and relatively easy to tear to any length by hand across the width, but at the same time should have sufficient longitudinal strength. CoFlex tapes and bandages (Andover Healthcare:, Salisbury, Mass.; see U.S. Pat. No. 3,575,782) may be suitably employ and have a laminated structure including two outer nonwoven layers with longitudinally-extending elastic yarns sandwiched between. POWERFLEX bandages (Andover Healthcare; see U.S. Pat. No. 5,762,623) also may be suitably employed as the bandage material and include a layer that is elastic in the longitudinally-extending direction laminated to one side of a warp-knitted, sometimes referred to as warp-knitted (weft insertion), fabric oriented with the knit yarns extending longitudinally.

In some embodiments, the bandage material is a “short stretch” elastic bandage and preferably a short stretch cohesive elastic bandage, which cohesively bonds to itself when wrapped in overlapping fashion, thus eliminating the need for bandage clips or other bandage fasteners. As used herein, the term “short stretch” refers to a bandage which is able to stretch, e.g., approximately 25% to 80% beyond its original, unstretched length. Unlike “long stretch” articles, which are able to stretch from approximately 100% up to several times beyond their original length in the longitudinal direction and thereby provide constant pressure at rest and work, i.e., a low static stiffness index (SSI), short stretch bandages are able to provide more effective compression through a low resting pressure and a high standing or working pressure, i.e., a high SSI, e.g., greater than about 10 mm Hg. The use of a low stretch compression bandage as the outer layer is especially advantageous for patients with venous insufficiency, since the low resting pressure provides comfort when the patient is recumbent while also preventing expansion of muscle diameter while the patient is ambulatory, thereby increasing venous and lymphatic return when the muscles of the leg contract.

In certain embodiments, the bandage material may be a latex-free cohesive elastic bandage, as disclosed in U.S. Pat. No. 6,156,424. In further embodiments, the bandage material may be a layer that eliminates the need for bandage scissors by facilitating hand tearing, as disclosed in U.S. Pat. No. 5,762,623.

One suitable bandage material is available from Andover Healthcare, Inc. (Salisbury, Mass.) under the trade designation “CoFlex NL.” CoFlex NL is intended for controlled compression that will not constrict over time, i.e., a short-stretch bandage.

Optionally, an additional outermost layer such as a stocking, tube, sleeve, or the like, preferably a nylon stocking, may be provided and worn over the wrapped bandage material, to provide a desired outward or cosmetic appearance of the bandage system.

Preferably, a zinc oxide composition and/or other one or more therapeutic agents are distributed throughout the bandage material. Such distribution can provide more extended application of calamine or other zinc oxide composition or other therapeutic agent(s) to the subject's skin around which the bandage material is wrapped.

Alternatively, a zinc oxide composition and/or other one or more therapeutic agents may be present only in selected regions of a bandage material, for example only within spaced strip areas that extend across the width and/or along the length of the bandage material. In one specific design, one or more polymer strips that contain a zinc oxide composition and/or other one or more therapeutic agents may be affixed (e.g. laminated) along the length or width of the bandage material.

A bandage material may be readily prepared. For instance, a formed bandage material may be impregnated with a zinc oxide containing composition or a composition containing one or more other therapeutic agents by dipping the bandage material in a bath containing the zinc oxide containing composition. The dipped bandage material then be formed into a roll at any desired length, width, and winding tension.

A mixture of polymer and a zinc oxide containing composition or a composition containing one or more other therapeutic agents also may be prepared. See, for instance, Example 1 which follows. That mixture then may be used to form a stretchable bandage material that includes a zinc oxide containing composition or a composition containing one or more other therapeutic agents.

In one particular aspect, a compression bandage may include multi-layers, for example one or more woven or nonwoven textile layer(s), an inner elastic layer and a knit textile layer all saturated and bound with a cohesive formula impregnated with a zinc oxide composition such as calamine and/or one or more other therapeutic agents as disclosed herein. In a preferred embodiment, the woven or nonwoven layer is an 8-10 gram per square meter, spun bound material such as polypropylene, polyethyleneteraphthalate or other material. Polypropylene is often preferred. Other polymeric woven or nonwovens may also be suitable. Although the preferred weight is 8-10 grams per square meter, other weights may be suitable. A preferred elastic layer may be comprised of transversely spaced, longitudinally extending coalesced multifilament elastic polyurethane strands such as Lykra or Spandex. In one preferred aspect, suitably the denier of the yarns is 210 and the spacing is 5-15 per inch. Other deniers and spacing also may be suitable. The knit textile layer suitably is a warp-knitted (weft insertion) polyester textile oriented with the knit yarns extending longitudinally and generally parallel to the orientation of the elastic strands. Other polymeric or natural fiber warp knits may be suitable as well. The warp-knitted (weft insertion) textile suitably has a weight of less than about 50 grams per square meter (e.g., about 1.5 oz. per square yard) and most preferably less than about 25 to 30 grams per square meter (about 0.7 to 0.9 oz. per square yard). Preferred fibers include those that are 12×12 fibers per inch, although other constructions may be suitable. The layers described above are then laminated with a cohesive formula that includes a zinc oxide composition and/or one or more other therapeutic agents as disclosed herein and metered to the desired coat weight. The cohesive formula's base polymer suitably can be, for example, natural rubber latex, polychloroprene or polyurethane. In the formula, there suitably may be one or more tackifying resins, surfactants, fillers, cross linkers, pigments, thixotropes, preservatives, etc. The zinc oxide composition such as calamine and/or one or more other therapeutic agents as disclosed herein are suitably added to the cohesive formula. Such incorporating of the zinc oxide composition and/or one or more other therapeutic agents can result in the zinc oxide composition and/or other therapeutic agent(s) being substantially homogenous throughout the bandage material. As a consequence, in preferred aspects, the bandage material can be wound on the patient in either direction onto the skin. Preferred resulting composite bandages can have a good appearance and hand-tear cleanly transversely across the bandage.

The following non-limiting examples are illustrative of the invention.

### EXAMPLE 1

A suitable compression bandage with added agent (calamine) can be prepared as follows as set forth in the following Examples 1 and 2.

Begin by adding polychloroprene dispersion (57.050 lb) to a clean vessel. When the polychloroprene reaches the bottom of the blade, turn on the mixer. Add Serdas 7005 defoamer (0.011 lb). Finish adding the balance of the polychloroprene. Add Snowtack 880G-rosin ester (13.910 lbs). Add Aquatac 6085 rosin ester (21.720 lbs) through an 800-micron filter. Add Bostex 24 antioxidant (1.086 lbs). Add Rovene 4301 carboxylated styrene butadiene (0.434 lbs). Sift in Calamine (5.625 lbs) using hopper or hand sifting. Add pigments per color specification. Red (0.043 lb) and neon yellow (0.130 lb) for Calamine color. Continue mixing until use to form the stretchable bandage material.

### EXAMPLE 2

A preferred compressions bandage system is provided by an 8-10 gram per square meter, spun bound polypropylene nonwoven with an elastic layer of transversely spaced, longitudinally extending coalesced multifilament Lykra or Spandex. The denier of the yarns is 210 and the spacing is 5-15 per inch. The knit textile layer is a warp-knitted (weft insertion) polyester textile oriented with the knit yarns extending longitudinally and generally parallel to the orientation of the elastic strands. The warp-knitted (weft insertion) textile has a weight of less than about 50 grams per square meter (about 1.5 oz. per square yard) and most preferably less than about 25 to 30 grams per square meter (about 0.7 to 0.9 oz. per square yard). The preferred fibers are 12×12 fibers per inch. The layers described above are then laminated with a cohesive formula described in Example 1 above and metered to the desired coat weight.

1. A compression bandage comprising:
a stretchable, wrappable bandage material for contact on a subject's skin,
wherein the bandage material comprises a zinc oxide composition.

2. The compression bandage of claim 1 wherein the bandage material comprises calamine.

3. A compression bandage comprising:
a stretchable, wrappable bandage material for contact on a subject's skin,
wherein the bandage material comprises one or more therapeutic agents.

4. The compression bandage of claim 3 wherein the bandage material comprises one or more of an antibiotic, an antibacterial agent, an antimicrobial agent, an anti-odor agent, an anti-inflammatory agent, an anti-fungal agent, an anti-viral agent, an anti-cancer agent, an eczema or psoriasis treatment agent, a pain relieving agent, or combination thereof.

5. The compression bandage of claim 4 wherein the bandage material comprises one or more of aloe; menthol; mupirocin; witch hazel, tree tea oil; clindamycin; anthralin; clotrimazole; ketoconazole; terbinafine; benzoyl peroxide; coal tar; corticosteroids; crisaborole; retinoids; salicylic acid; acyclovir; 5-fluorouracil; 5-fluorouracil, diclofenac; hyaluronic acid; imiquimod; ingenol mebutate; adalimumab; and eucrisa.

6. The compression bandage of claim 3 wherein the bandage material further comprises a zinc oxide composition.

7. The compression bandage of claim 6 wherein the zinc oxide composition and/or therapeutic agents are distributed substantially uniformly throughout the bandage material.

8. The compression bandage of claim 6 wherein the zinc oxide composition and/or therapeutic agents reside in selected regions of the bandage material.

9. The compression bandage of claim 6 wherein the zinc oxide composition and/or therapeutic agents are present in first regions of the bandage material and are not present in second regions of the bandage materials which are distinct from the first regions.

10. The compression bandage of claim 3 wherein the bandage has as an unstretched length and a maximum stretched length, wherein the maximum stretched length is about 40-80% or more greater than the unstretched length.

11. The compression bandage of claim 3 wherein the bandage is stretchable to provide a compressive force when wrapped about the limb of a subject.

12. The compression bandage of claim 3 wherein the bandage material comprises latex.

13. The compression bandage of claim 3 wherein the bandage material is latex-free.

14. The compression bandage of claim 3 wherein the bandage material is in a roll.

15. The compression bandage of claim 3 wherein the bandage material is between 1 and 6 inches wide and rolled along the bandage material length.

16. A method for treating a subject comprising:
wrapping the compression bandage of claim 3 around a subject, wherein the bandage directly contacts skin of the subject.

17. The method of claim 16 wherein the compression bandage is wrapped around a limb, torso or head of a subject.

18. The method of claim 16 wherein the compression bandage applies a compressive force to the subject.

19. The method of claim 16 wherein the zinc composition and/or one or more therapeutic agents is administered transdermally to the subject.

20. The method of claim 16 wherein the compression bandage is not utilized with another separate bandage material.

21. The method of claim 16 further comprising applying a distinct bandage or protective layer over the wrapped bandage material.

22. The method of claim 21 wherein the distinct bandage or protective layer is a sleeve or stocking.

23. The method of claim 16 wherein the subject is identified as suffering from a disease or disorder that may be treated with the compression bandage.

24. The method of claim 16 wherein the subject is identified as suffering from a skin cancer or a skin pre-cancer.

25. The method of claim 16 wherein the subject is identified as suffering from hypertrophic solar keratoses, actinic keratosis and/or squamous cell carcinoma.

26. The method of claim 16 wherein the compression bandage comprises one or more of 5-fluorouracil, diclofenac and/or hyaluronic acid, imiquimod and ingenol mebutate.

27. A kit comprising:
(a) the compression bandage of claim 3, and
(b) written instructions for use and/or identifying information of the compression bandage.

28. The kit of claim 27 further comprising (b) written instructions for use and/or identifying information of the compression bandage.

29. The kit of claim 27 wherein the compression bandage is in roll form.

30. The kit of any one of claims 27 through 29 wherein the compression bandage is in roll form.

31. The kit of any one of claims 27 through 30 wherein the kit does not contain a separate, additional bandage material.

32. A kit that comprises 1) a box unit, 2) a roll of the compression bandage of claims 3, and 3) written instructions for use and/or identifying information of the compression bandage.

33. The kit of any one of claims 26 through 30 wherein the kit comprises within sealed packaging: 1) a roll of the compression bandage and 2) written instructions for use and/or identifying information of the compression bandage.

34. The kit of any one of claims 26 through 32 wherein the kit consists essentially of 1) a roll of the compression bandage and 2) written instructions for use and/or identifying information of the compression bandage.

35. The kit of any one of claims 26 through 32 wherein the kit consists of 1) a roll of the compression bandage and 2) written instructions for use and/or identifying information of the compression bandage.
